(19) United States
(12) Patent Application Publication
(10) Pub. No.: US 2006/0229658 A1
(43) Pub. Date: Oct. 12, 2006

(54) Embolic protection filter with reduced landing zone

(76) Inventor: Timothy M. Stivland, Plymouth, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/100,858

(22) Filed: Apr. 7, 2005

(51) Int. Cl. A61M 29/00

(52) U.S. Cl. 606/200

(57) ABSTRACT

An emboli capturing filter device and system are provided. The filter device includes an expandable filter disposed about an elongate member. The filter has a proximal mouth portion facing the proximal end of the elongate member, and a distal portion extending toward to distal end of the elongate member. A support arm is coupled to the elongate member and mouth portion of the expandable filter. When the filter is in an expanded orientation, the support arm is attached to the elongate member at or distal of the mouth of the filter.

### BACKGROUND

The present invention is an emboli capturing system. More specifically, the present invention involves an emboli capturing system and method for capturing embolic material in a blood vessel during an atherectomy or thrombectomy procedure.

Blood vessels can become occluded (blocked) or stenotic (narrowed) in one of a number of ways. For instance, a stenosis may be formed by an atheroma which is typically a harder, calcified substance which forms on the lumen walls of the blood vessel. Also, the stenosis can be formed of a thrombus material which is typically much softer than an atheroma, but can nonetheless cause restricted blood flow in the lumen of the blood vessel. Thrombus formation can be particularly problematic in a saphenous vein graft (SVG).

Two different procedures have been developed to treat a stenotic lesion (stenosis) in the vasculature. The first is to deform the stenosis to reduce the restriction within the lumen of the blood vessel. This type of deformation (or dilatation) is typically performed using balloon angioplasty.

Another method of treating stenotic vasculature is to attempt to completely remove either the entire stenosis, or enough of the stenosis to relieve the restriction in the bloods vessel. Removal of the stenotic lesion has been done through the use of radio frequency (RF) signals transmitted via conductors and through the use of lasers, both of which treatments are meant to ablate (i.e., super heat and vaporize) the stenosis. Removal of the stenosis has also been accomplished using thrombectomy or atherectomy. During thrombectomy and atherectomy, the stenosis is mechanically cut or abraded away from the vessel.

Certain problems may be encountered during thrombectomy and atherectomy. The stenotic debris which is separated from the stenosis is free to flow within the lumen of the vessel. If the debris flows distally, it can occlude distal vasculature and cause significant problems. If it flows proximally, it can enter the circulatory system and form a clot in the neural vasculature, or in the lungs, both of which are highly undesirable. Angioplasty may also result in release of debris.

Prior attempts to deal with the debris or fragments have included cutting the debris into such small pieces (having a size on the order of a blood cell) that they will not occlude vessels within the problems. It is difficult to control the size of the fragments of the stenotic lesion which are severed, and larger fragments can be severed accidentally. Also, since thrombus is much softer than an atheroma, it tends to break up easier when mechanically engaged by a cutting instrument. Therefore, at the moment that the thrombus is mechanically engaged, there is a danger that it can be dislodged in large fragments which could occlude the vasculature.

Another attempt to deal with debris severed from a stenosis is to remove the debris as it is severed using suction. It may be necessary to pull quite a high vacuum in order to remove all of the pieces severed from the stenosis. If a high enough vacuum is not used, all of the severed pieces will not be removed. However, the use of a high vacuum may cause the vasculature to collapse.

A final technique for dealing with the fragments of the stenosis which are severed during atherectomy is to place a device distal to the stenosis during atherectomy to catch the pieces of the stenosis as they are severed, and to remove those pieces along with the capturing device when the atherectomy procedure is complete. Such capture devices have included expandable filters which are placed distal of the stenosis to capture stenosis fragments.

One limitation of distal embolic protection is the space required between the lesion to be treated and the filter component. This is particularly important when a lesion is near a bifurcation such as the distal anastomosis of a vein graft or a major side branch in native coronary arteries. For example, some devices require 3 cm or more from the lesion to the filter component due to structural components of the device. This eliminates 25-30% of potential saphenous vein graft cases.

### SUMMARY

An emboli capturing system that captures emboli adjacent a lesion in a body lumen is provided. An expandable emboli capturing device is mounted proximate the distal end of an elongate member, and is movable between a radially expanded position and a radially contracted position. When in the expanded position, the emboli capturing device forms a basket or net with a proximally opening mouth. The device is configured such that the mouth can be positioned adjacent a lesion to be treated.

The embolic protection device includes an expandable filter disposed about the elongate member and a support arm with a first end coupled to the elongate member and a second end coupled to the mouth portion of the expandable filter. When the filter is in an expanded orientation, the first end of the support arm is disposed at or distal of the mouth of the filter. The filter is self-expanding and biased in the expanded orientation. In some embodiments, the support arm is slidingly disposed on the elongate member, which can be a guidewire. The expandable filter is supported at least at the mouth portion by a frame that defines the mouth of the filter. The support arm is attached to the frame. In some embodiments, the support arm is connected to the elongate member distal of the mouth of the filter. In other embodiments, the support arm is substantially perpendicular to the elongate member. The support arm can be expandable and retractable.

In another embodiment of the invention, at least one support arm is attached to the elongate member at an attachment point and attached to a frame disposed about the elongate member such that the frame is spaced from the elongate member. The proximal opening of a filter is attached to the frame, with the distal end of the filter extending towards the distal end of the elongate member. The attachment point is in substantially the same axial space as the proximal opening of the filter. In another embodiment, the attachment point is distal of the proximal opening of the filter member. The support arm can be attached to the elongate member via an attachment member, which can be slidingly disposed on the elongate member. The support arm can be moveable between a first position in which the attachment member is proximal of the frame, and a second position in which the attachment member is distal of the frame. In a further embodiment, the attachment member expands and contracts around the elongate member thereby reversibly holding and releasing the attachment member to the elongate member.

An emboli capturing system is also provided, including an expandable filter device disposed about an elongate member and a retrieval member configured to be longitudinally moveable over the elongate member. The retrieval member has a receiving end configured to receive the filter in a collapsed position. The filter device has a proximal mouth portion facing the proximal end of the elongate member, a distal portion extending toward to distal end of the elongate member, and at least one support arm coupling the mouth portion of the filter. The expandable emboli capturing device is moveable between a radially expanded position and a radially collapsed position. When the filter is in an expanded orientation, the first end of the support arm is disposed at or distal of the mouth of the filter. In some embodiments, the retrieval member includes an inner member adapted to engage the support arm thereby collapsing the filter. The inner member can be a hollow tube. The support arm can be coupled to the elongate member by an attachment member, and the attachment member can include one or more fixing elements that mechanically engage the retrieval member.

### BRIEF DESCRIPTION OF THE DRAWINGS

FIG. 1 shows a distal protection device of the present invention in a deployed position.

FIG. 2 shows another distal protection device of the present invention in a deployed position.

FIG. 3 shows the distal protection device of FIG. 2 in a collapsed configuration prior to deployment.

FIG. 4 shows the distal protection device of FIG. 2 with a retrieval sheath over the arm.

FIG. 5 shows the distal protection device of FIG. 4 with the retrieval sheath collapsing the filter member.

FIG. 6 shows a distal protection device according to another embodiment of the invention after deployment and prior to movement of arm into the distal position.

FIG. 7 shows the distal protection device of FIG. 6 with the arm moved into the distal position.

FIG. 8 shows the distal protection device of FIG. 7 deployed distal of a treatment site, with a stent delivery device moving the arm distally.

FIG. 9 shows a balloon catheter and stent positioned adjacent a distal protection device with an “S” shaped arm.

FIG. 10 shows another embodiment of distal protection device with an “S” shaped arm.

FIG. 11 shows a distal protection device with a coil attachment member.

FIG. 12 shows a distal protection device with an extended attachment member that fits over an existing guidewire.

FIG. 13 shows a distal protection device with a hollow attachment member having a retainer ring.

FIG. 14 shows a distal protection device with an expanding and contracting attachment member.

FIG. 15 shows a distal protection device and a retrieval member with a pusher.

FIG. 16 shows a distal protection device with an elongated hollow retrieval member.

FIGS. 17A and 17B show a distal protection device with a retractable arm.

FIG. 18 shows a collapsible or foldable spinner tube.

FIG. 19 shows a distal protection device with a filter distal of the distal end of the elongate member.

FIG. 20 shows a distal protection device with an attachment member and arm moveable between a proximal position and a distal position.

FIG. 21 shows a distal protection device with a tether connected to the arm.

FIG. 22 shows a distal protection device with a bent arm.

FIG. 23 shows a distal protection device with a split spinner tube as the attachment member.

FIG. 24 shows a distal protection device with a spring arm

FIG. 25 shows a distal protection device with a second arm attached to the elongate member distal the end of the filter.

FIG. 26 shows a distal protection device with a sliding attachment member.

FIG. 27 shows a distal protection device with a spiral arm.

FIG. 28 shows a distal protection device with an attachment member having fixing elements.

FIG. 29 shows a distal protection device with a tether attached to the frame.

FIG. 30 shows a distal protection device with two arms and two attachment members, one proximal and one distal.

### DETAILED DESCRIPTION

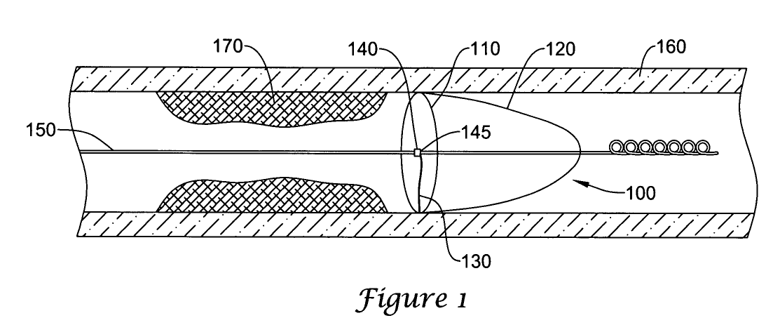

FIG. 1 illustrates a filter device 100 in place downstream of a lesion or stenosis 170 in a vessel 160. The filter device 100 has a hoop-shaped frame 110 supporting filter member 120 in an expanded and deployed position. The filter device 100 includes arm 130 extending from the frame 110 to an attachment member 140 disposed on an elongate member 150 at attachment region 145. The arm 130 connects frame 110 to elongate member 150 through an attachment member 140. The attachment member 140 can be fixed to the elongate member 150 or it can be slidably connected. The configuration of the arm 130, frame 110 and elongate member 150 provides some additional structural integrity to frame 110, while allowing frame 110 to substantially float about elongate member 150 in the region of frame 110. The configuration of the filter device 100 is such that it can be deployed close to the distal edge of the stenosis 170.

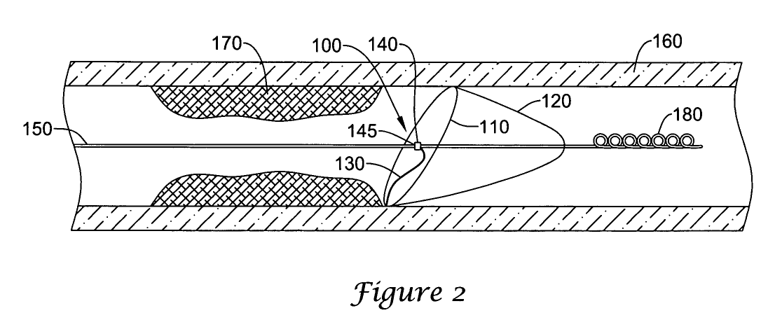

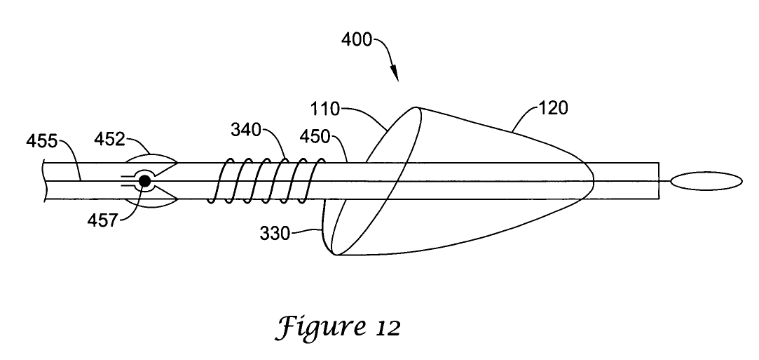

FIG. 1 illustrates arm 130 extending substantially vertically from frame 110 to attachment member 140. The angles at which the arm 130 is attached to the frame 110 and to the attachment member 140 can be varied to alter the angle of the mouth of the filter device 100 with respect to the vessel walls. The mouth and frame 110 of filter device 100 shown in FIG. 1 are substantially perpendicular to the vessel 160. The mouth of the filter device 100 can be tilted backward, in which an arbitrary “top” of the frame 110 is angled toward the distal end of the elongate member 150. See FIG. 2. Alternatively, the device can be tilter forward with the “top” of the frame 110 angled toward the proximal end of the elongate member 150. See FIG. 12.

FIG. 2 shows a filter device 100 according to another embodiment of the invention. The filter device 100 is deployed distal of stenosis 170 within the lumen of a blood vessel 160. Filter device 100 includes a frame 110, filter member 120, arm 130, attachment member 140, and elongate member 150. Filter device 100 is deployed adjacent stenosis 170 and is oriented such that filter member 120 opens toward the proximal end of elongate member 150 to catch embolic material released from the stenosis 170.

In some embodiments, the frame 110 is formed of a material having some shape memory. Thus, when frame 110 is collapsed for deployment, it collapses about the elongate member 150, and then expands to the open configuration shown in FIG. 2 upon deployment. Frame 110 can be made of an expandable material such as an expandable polymer or metal or other elastic material. In one embodiment, frame 110 is a self-expanding hoop formed of a wire that includes a shape memory alloy. In another embodiment, hoop-shaped frame 110 is formed of a nitinol wire having a diameter in a range of approximately 0.002-0.004 inches. Frame 110 is biased in an expanded configuration. Properties of nitinol are used to form a frame at least in the area of the mouth of the distal protection filter. Thus, the distal protection device can be deployed, retrieved, and re-deployed any number of times without incurring plastic deformation.

The distal end of elongate member 150 can be connected to a coil tip 180. In one embodiment, coil tip 180 is brazed or otherwise welded or suitably connected to the distal portion of elongate member 150. In some embodiments, elongate member 150 is a wire such as a guidewire. In other embodiments, elongate member 150 is a conventional stainless-steel guidewire having conventional guidewire dimensions. For instance, in one embodiment, elongate member 150 is a solid core wire having an outer diameter of approximately 0.014 inches and an overall length of up to 300 cm.

It will be noted that other suitable guidewire dimensions and configurations can also be used. For example, guidewires having an outer diameter of approximately 0.018 inches may also be used. For other coronary applications, different dimensions may also be used, such as outer diameters of approximately 0.010 inches to 0.014 inches. Further, it will be appreciated that the particular size of elongate member 150 will vary with application. Applications involving neural vasculature will require the use of a smaller guidewire, while other applications may require the use of a larger guidewire. In some embodiments, elongate member 150 is formed of stainless steel. In other embodiments, elongate member 150 is a hollow guidewire or hypotube 350.

In some embodiments, it may be desired to make elongate member 150, frame 110, and/or filter member 120 radiopaque. Radiopaque loaded powder can be used to form a polyurethane sheath which is fitted over elongate member 150 or frame 110, or which is implemented in filter member 120. Alternatively, frame 110 and elongate member 150 can be gold plated in order to increase radiopacity. In other embodiments, marker bands are disposed on elongate member 150 or filter member 120 to increase the radiopacity of the device.

By connecting frame 110 to elongate member 150 through arm 130, elongate member 150 is spaced apart from frame 110. In this configuration, frame 110 can follow the vasculature without kinking or prolapsing (i.e., without collapsing upon itself). Thus, certain positioning or repositioning of filter member 120 can be accomplished with less difficulty.

The configuration of the arm 130 and its position with respect to frame 110 and the mouth of the filter device 100 allow the filter device 100 to be disposed adjacent a lesion to be treated. The prior distal filters generally require a distance of about 3 cm between the stenosis and the mouth of the filter due to the structure of the filter and its supporting legs or struts. See FIG. 1. The location of the filter with respect to the lesion, also known as the landing zone, limits the situations in which the filter can be used. For example, filter devices having a landing zone of 3 cm or greater are not suitable for use when a lesion is near a bifurcation such as the distal anastomosis of a vein graft or a major side branch in native coronary arteries. In these situations, a filter must be capable of being placed adjacent the stenosis, with little or no space between the lesion and the mouth of the filter. This reduced landing zone feature is achieved with the filter device 100 of the invention.

In some embodiments, arm 130 is a wire. Arm 130 may be made of a shape memory material such as nitinol, or a high tensile, flexible material such as KEVLAR®. Arm 130 can also be formed of an appropriate polymer material. In some embodiments, arm 130 has a rigidity or stiffness sufficient to maintain the filter device 100 in the desired position. In other embodiments, arm 130 can be flexible, and the length of the arm 130 maintains the filter device 100 in the desired position. Arm 130 can be a crescent-shaped solid, or it can be formed of two or more wires connected at their ends to the frame 110 and attachment member 140. Arm 130 can be shaped with an appropriate curvature to facilitate apposition of the frame to the vessel wall and recovery by the retrieval member. In some embodiments, arm 130 is attached to elongate member 150 at attachment region 145 by soldering, welding, brazing, or other heat set fixing means, adhesive, or any other suitable attachment mechanism.

In other embodiments, arm 130 is attached to an attachment member 140 that is disposed on elongate member 150. In some embodiments, attachment member 140 is fixed to elongate member 150, and in other embodiments attachment member 140 is slidable or moveable along elongate member 150. The degree and ease of movement of the attachment member 140 along elongate member 150 varies according to the deployment and retrieval mechanisms. In alternative embodiments, the attachment member 140 can be adapted to slide along or be fixed to an existing guide wire as the elongate member. In a further embodiment, modular filter devices may include an element that fits over an existing guide wire. The filter member is attached to the element via an arm, with or without an attachment member. The element is releasably connected to the guide wire by adhesive, compression fitting, friction fit, or any other suitable connection means.

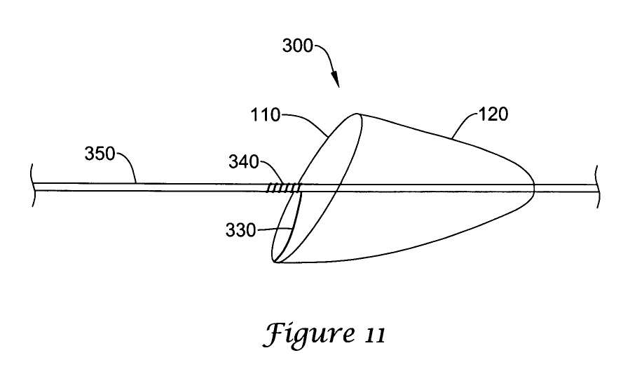

In the embodiment shown in FIG. 2, attachment member 140, formed of a weldable material, is attached to arm 130. The attachment member 140 is then attached to elongate member 150 with adhesive, welding, brazing, heat, or any other suitable attachment means. In the filter device 300 shown in FIG. 11, arm 330 is attached to elongate member 350 by a coil 340. Arm 330 and coil 340 can be formed of a single length of wire. The wire is attached at one end to frame 110, extends to and is wrapped around elongate member 350, forming coil 340. Alternatively, arm 330 and coil 340 may be separately formed and may be made of different materials. In the filter device 300 shown in FIG. 11, the coil 340 extends proximally from the point of attachment to arm 330. Alternatively, the coil 340 can be wound such that it extends distally towards the filter member 120. The windings of the coil 340 can be close together or spaced apart and may be tightly wound around the elongate member 150 loosely wound to allow axial movement along the elongate member 150.

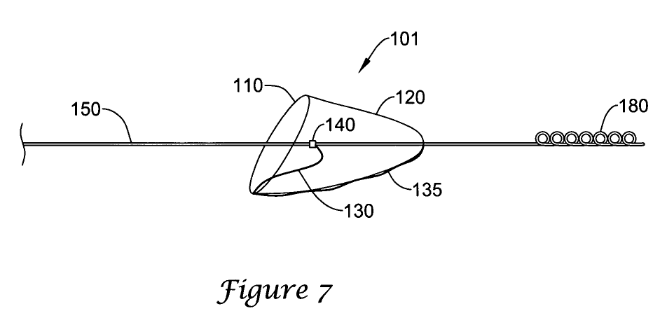

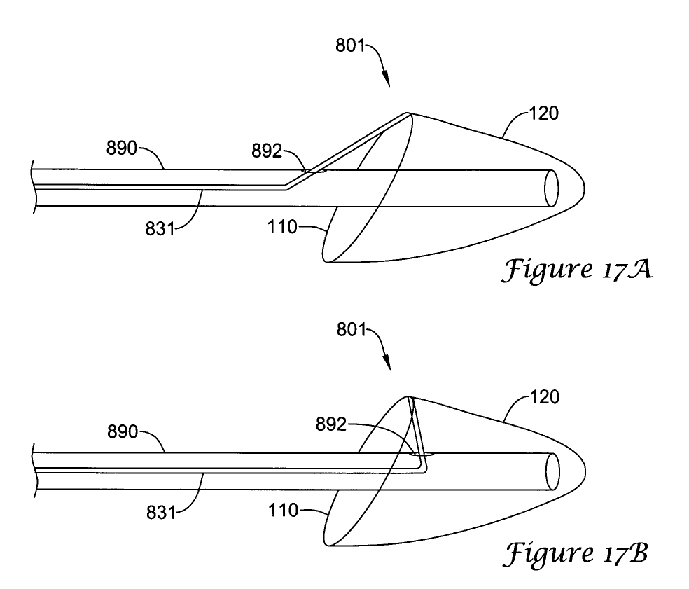

Arm 130 and frame 110 can be in substantially the same axial space, as shown in FIGS. 1, 2, 11 and 27. In other embodiments, arm 130 extends distally into the filter member 120 and is disposed on elongate member 150 distal of frame 110, as shown in FIGS. 7, 9, and 10. Arm 130 can also be moveable between proximal and distal positions, as shown in FIGS. 17A-17B, 20, and 24.

Filter member 120 is a microporous membrane, or other suitable mesh or perforated material that forms a substantially lumen-filling filter that allows blood to flow therethrough, but that provides a mechanism for receiving and retaining stenosis fragments carried into filter member 120 by blood flow through the vessel 160. Filter member 120 forms a generally conical basket opening toward the proximal end of elongate member 150. In some embodiments, filter member is formed of woven or braided fibers or wires, or a microporous membrane, or other suitable filtering or netting-type material.

In one embodiment, filter member 120 is a microporous membrane having holes therein with a diameter of approximately 100 μm. Filter member 120 can be disposed relative to frame 110 in a number of different ways. For example, filter member 120 can be formed of a single generally cone-shaped piece which is secured to the outer or inner periphery of frame 110. Alternatively, filter member 120 can be formed of a number of discrete pieces which are assembled onto frame 110.

In some embodiments, filter member 120 is formed of a polyurethane material having holes therein such that blood flow can pass through filter member 120, but emboli (of a desired size) cannot pas through filter member 120 and are retained therein. In one embodiment, filter member 120 is attached to hoop-shaped frame 110 with a suitable commercially available adhesive. In another embodiment, filter member 120 has a proximal portion thereof folded over hoop-shaped frame 110, and the filter material is attached to itself either with adhesive, by stitching, or by another suitable connection mechanism, in order to secure it about hoop-shaped frame 110. This connection can be formed by a suitable adhesive or other suitable connection mechanism.

In some embodiments, the distal end of filter member 120 is attached about the outer periphery of elongate member 150, proximate coil tip 180. In one configuration, filter member 120 is approximately 15 mm in longitudinal length, and has a diameter at its mouth (defined by hoop-shaped frame 110) of a conventional size (such as 4.0 mm, 4.5 mm, 5 mm, 5.5 mm, or 6 mm). It will be noted that any other suitable size can be used as well. In further embodiments, filter member 120 is formed of a polyurethane material with holes laser drilled therein. The holes can be approximately 100 μm in diameter. Filter member 120 can also be a microporous membrane, a wire or polymer braid or mesh, or any other suitable configuration.

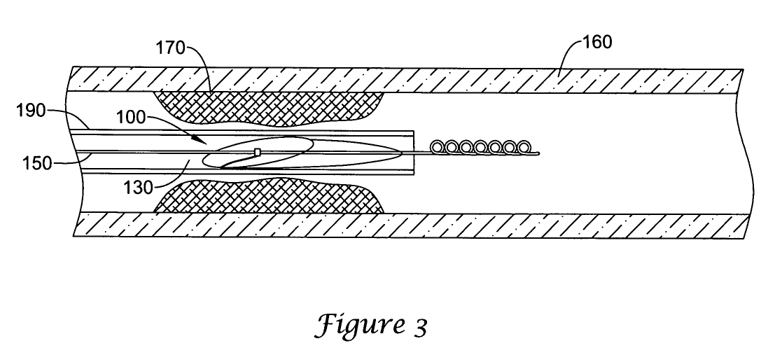

The filter device 100 is delivered in a collapsed configuration inside a delivery sheath or sleeve 190. In operation, frame 110 and filter member 120 are collapsed to a radially contracted position against elongate member 150 within delivery sleeve 190, as shown in FIG. 3. Sleeve 190 slides over elongate member 150 and is sized to fit around the outer periphery of expandable frame 110 when expandable frame 110 is in the collapsed position. Elongate member 150 is manipulated to position filter device 100 distal of a lesion 170 to be treated. FIG. 3 illustrates filter device 100 in the collapsed configuration in delivery sleeve 190 prior to deployment. Sleeve 190 is withdrawn proximally over elongate member 150. Once filter device 100 is no longer restrained by sleeve 190, filter device 100 assumes its expanded shape memory position in the vasculature as illustrated in FIG. 2. Frame 110 self-expands radially outwardly from the outer surface of elongate member 150, depositing the proximal mouth of filter member 120 against the vessel walls.

Filter device 100 forms a substantially lumen-filling basket or filter which allows blood to pass distally therethrough, but which retains or captures embolic material carried by the blood flow. The physician then simply removes sleeve 190 from the vasculature leaving filter device 100 in place during subsequent procedures. A suitable treatment device is then advanced over elongate member 150 and is used to compress, sever, fragment, or otherwise treat the vascular restriction or lesion 170. Emboli are carried by blood flow distal of the restriction are captured by filter member 120. After the treatment procedure, filter member 120, along with the emboli retained therein, are retrieved from the vasculature. Various retrieval procedures and devices are described later in the specification.

It should be noted that the stenosis removal device (or atherectomy catheter) used to fragment stenosis 170 can be advanced over elongate member 150. Therefore, the device according to the present invention is dual functioning in that it captures emboli and does not require adding an additional device to the procedure. Instead, the present invention simply replaces a conventional guidewire with a multi-functional device.

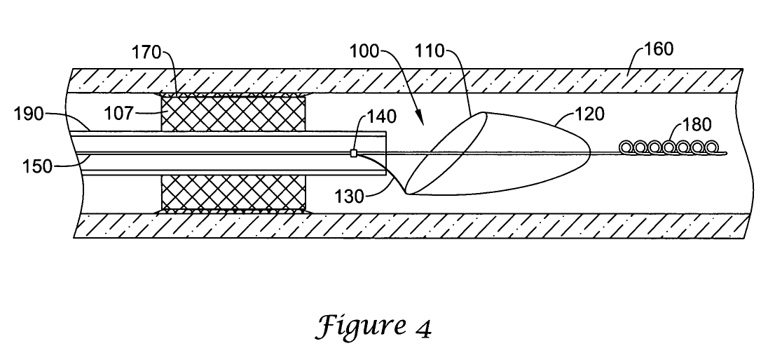

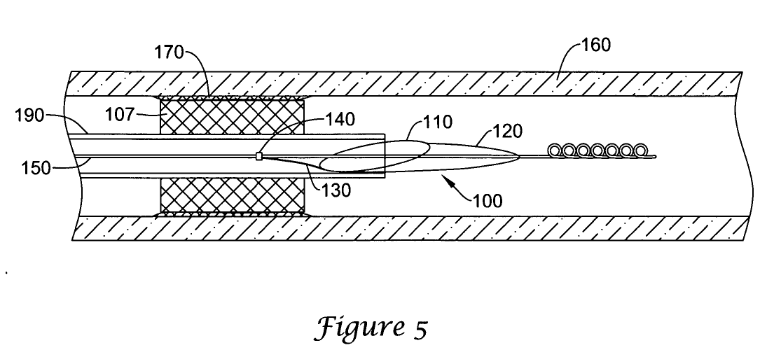

FIGS. 4 and 5 illustrate retrieval of the filter device 100 by advancing sleeve 190 distally over elongate member 150. Sleeve 190 passes over attachment member 140 and arm 130, urging the arm 130 closer to elongate member 150 and tilting the frame 110 backward. As sleeve 190 passes over the attachment point between the frame 110 and arm 130, frame 110 collapses against elongate member 150 as the filter device 100 is pulled into sleeve 190.

The following embodiments include a filter member, frame, elongate member, arm, and attachment member similar to those discussed above. The configuration of the arm, frame, and attachment member allow for the mouth of the filter member to be disposed adjacent the lesion to be treated, achieving a distal protection filter with a reduced landing zone.

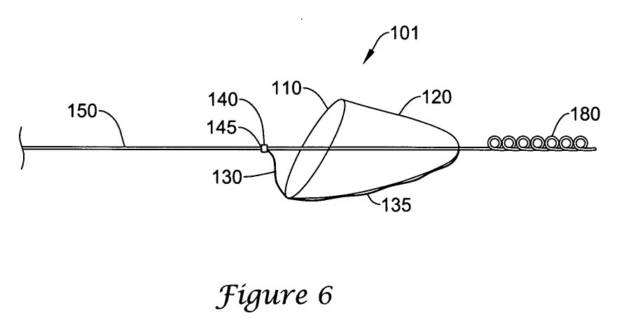

FIGS. 6-8 illustrate a filter device 101 in which the arm 130 is adjustable from a first, proximal position, as shown in FIG. 6, to a second, distal position as shown in FIG. 7. The filter also includes a flexible member 135 that extends from the frame 110 distally along the inside of the filter member 120. In some embodiments, the flexible member 135 extends all the way to the distal end of the filter member 120. In other embodiments, the flexible member 135 extends part way toward the distal end of the filter member 120. The flexible member 135 is attached to the frame 110 and can be attached along its length or at discrete locations to the filter. In a further embodiment, a plurality of flexible members 135 are attached to the frame 110 and extend distally along the filter member 120. In some embodiments the flexible member 135 is made of a shape memory material such as nitinol. The flexible member 135 aids in delivery and expansion of the filter member 120 by urging the filter member 120 away from the elongate member 150 as the frame 110 is released from a delivery sleeve.

The arm 130 can be made of a high tensile, flexible material such as KEVLAR® that permits the movement between the first and second positions. In another embodiment, the arm 130 can be attached to frame 110 by a hinge, pivot, or other structure that facilitates movement of the arm 130 to a desired position relative to the frame 110. In a still further embodiment, the hinge, pivot, or other structure permits the arm 130 to be in either the first or second position, but not in an intermediate position.

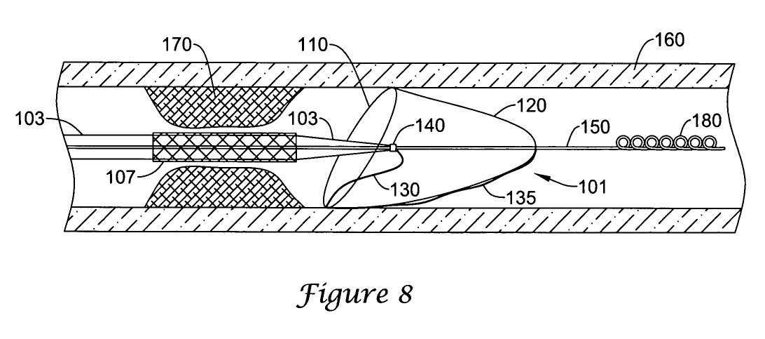

FIG. 8 illustrates a stent delivery device 103 that also functions to position arm 130 of filter device 101. The filter device 101 is deposited within the vessel 160 distal of stenosis 170. The arm 130 is in the first position, as shown in FIG. 6, proximal of the frame 110. The stent delivery device 103 carries a stent 107 and has a distal end configured to move the attachment member 140 of filter device 101. The stent delivery device 103 can have an inflatable or expandable region for expanding the stent. The stent delivery device 103 is adapted to be advanced over elongate member 150. The stent delivery device 103 is moved distally towards the filter device 101, the distal end contacts and moves the attachment member 140 distally into the filter member 120. The movable arm 130 allows the filter device 101 to be positioned very close to the stenosis 170 while still allowing a stent delivery device to advance a sufficient distance toward the filter device 101 for placement of the stent 107. The mouth of the filter device 101 can be positioned 0.5 to 1.0 cm from the distal end of the lesion. In some embodiments, the filter device 101 can be placed less than 0.5 cm from the distal end of the lesion.

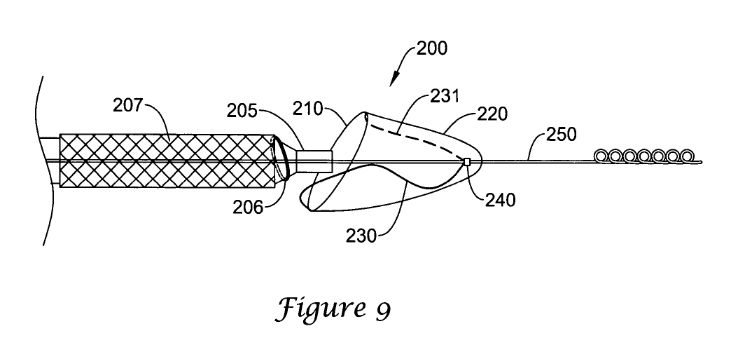

The filter device 200 shown in FIG. 9 includes filter member 220, frame 210, and elongate member 250. Arm 230 is biased in an “S” configuration that telescopes and contracts with longitudinal movement of the filter device 200 with respect to elongate member 250. This feature allows the distal end of a stent delivery device, such as the balloon catheter 207 shown in FIG. 9 to be advanced into the mouth of the filter device 200 during stent deployment without dislodging the filter device 200 from the vessel. As shown in FIG. 9, when the balloon catheter 207 is advanced over elongate member 250, the distal end 205 of the catheter with a balloon stop 206 moves into the mouth of the filter device 200 and contacts arm 230. The arm 230 can straighten, pushing attachment member 240 distally towards the distal end of the filter member 220, while the frame 210 remains seated against the vessel walls, keeping the filter device 200 in position. Once the stent has been deployed and the balloon catheter 207 is withdrawn, arm 230 returns to its resting “S” shaped configuration.

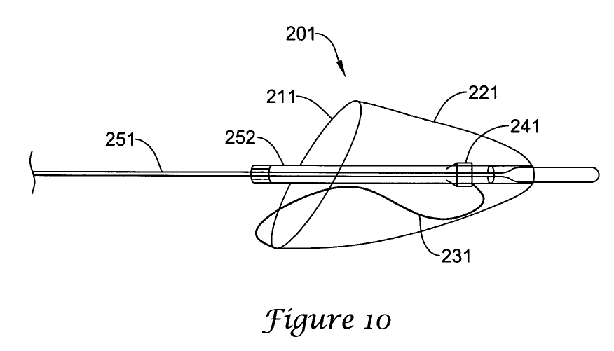

FIG. 10 illustrates a filter device 201 having a frame 211, filter member 221, retractable arm 231, elongate member 251, and slidable member 252. The slidable member 252 is disposed on the elongate member 251 and contains the attachment member 241, to which the arm 231 is connected. As slidable member 252 slides along elongate member 251, arm 231 is expanded or contracted.

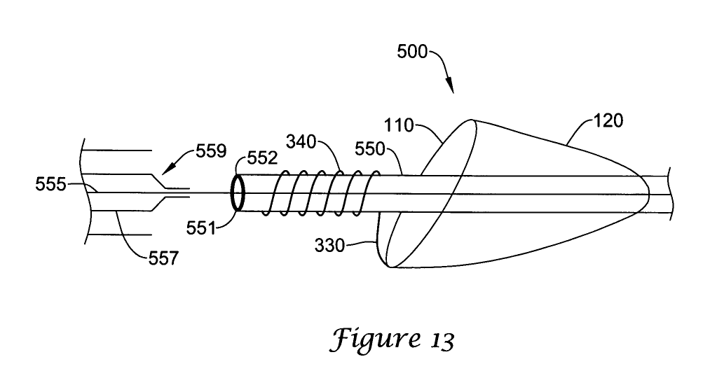

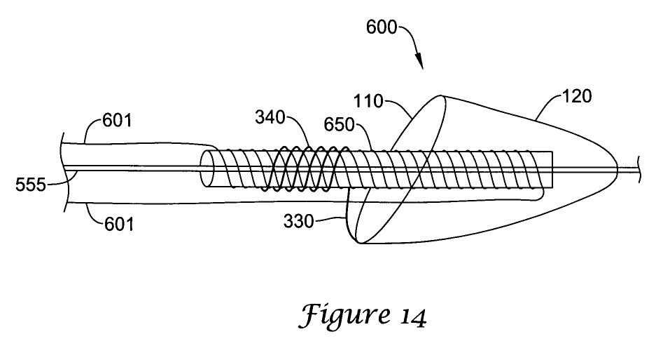

In a further embodiment, shown in FIG. 12, modular filter device 400 includes filter member 120, frame 110, arm 330, attachment member 340, and hollow elongate member 450 with receiving member 452. The device 400 is configured to be secured to an existing guidewire 455 having a detent or bump 457. Receiving member 452 on elongate member 450 is configured to slide over and receive detent or bump 457 on guidewire 455. In an alternate embodiment, shown in FIG. 13, hollow elongate member 550 has an expandable retainer ring 552 at its proximal end 551. In use, the modular filter device 500 is loaded onto a conventional guidewire 555 using ring expander 557. Ring expander 557 has a tapered or angled distal end 559 and slides over guidewire 555. Tapered or angled distal end 559 fits into expandable retainer ring 552, expanding ring 552. Expandable retainer ring 552 is elastic and grips ring expander 557 when expanded. Another embodiment of modular filter device 600, shown in FIG. 14, includes electrically actuated elongate member 650 that expands and contracts with electrical current to grip and release guidewire 555. Electrical contacts 601 provide the current to expand and contract elongate member 650. The electrically actuated elongated member 650 can be a bi-metal or other electrically actuated coil.

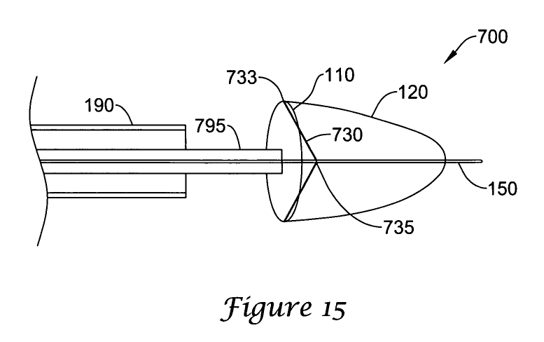

The filter device 700 illustrated in FIG. 15 has one or more arms 730 that are fixed at a first end 733 to frame 110 and are slidably attached to elongate member 150 at a second end 735. Arms 730 are rigid and extend substantially perpendicular from elongate member 150 when the filter device 700 is in a deployed configuration. During retrieval, a retrieval sheath or sleeve 190 with pusher 795 is advanced over elongate member 150 to just proximal the filter device 700. Pusher 795 is extended from within sleeve 190, until it contacts arms 730 and slides the second ends 735 of arms 730 in a distal direction, thereby collapsing frame 110 and filter member 120 about the elongate member 150. Sleeve 190 is then advanced over collapsed filter device 700 for retrieval.

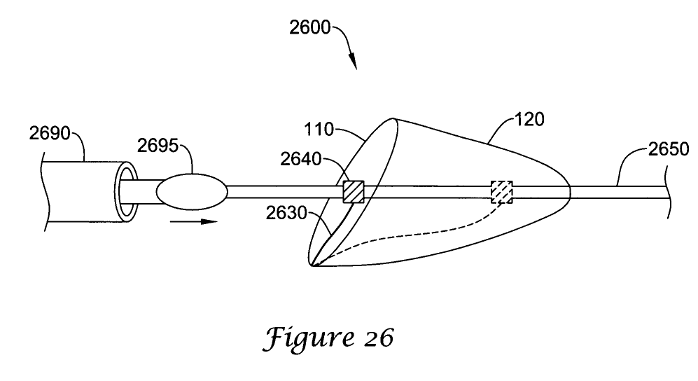

FIG. 26 illustrates an alternative embodiment, in which arm 2630 is attached to sliding attachment member 2640. During retrieval, retrieval sheath or sleeve 2690 containing pushing member 2695 is advanced distally over elongate member 2650 to the site of filter device 2600. Pushing member 2695 is advanced into filter member 120, moving arm 2630 distally, thereby collapsing filter device 2600 around elongate member 2650.

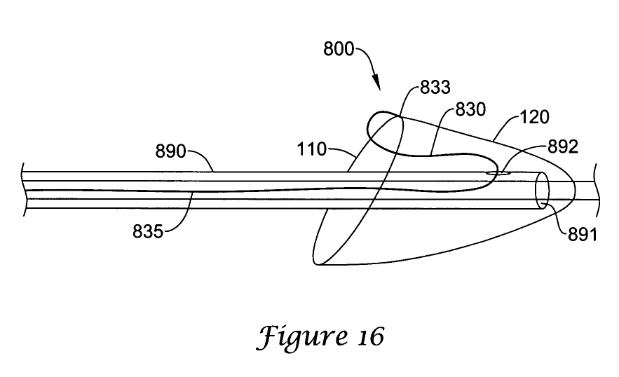

In the filter device 800 shown in FIG. 16, an “S” shaped arm 830 is attached at a first end 833 to frame 110. The second end 835 of arm 830 extends distally into filter member 120, into retrieval sleeve 890, and then proximally through retrieval sleeve 890. Arm 830 can enter sleeve 890 at distal end 891 of sleeve 890 or through an opening 892 in sleeve 890. In some embodiments, arm 830 is a wire. Alternatively, arm 830 can be a strand, thread, braid, or other elongate structure made of a flexible material. During retrieval, distally advancing the retrieval sleeve 890 collapses the filter device 800 against the sleeve 890.

An alternative filter device 801 is shown in FIGS. 17A and 17B. The filter device 801 has a retractable arm 831 instead of “S” shaped arm 830. Retractable arm 831 extends from frame 110 through opening 892 in sleeve 890. When sleeve 890 is moved distally, retractable arm 831 moves distally into filter member 120. Proximal movement of sleeve 890 pulls arm 831 away from filter member 120 and frame 110, thereby collapsing filter 120 and frame 110 against sleeve 890 for retrieval of the filter device 801.

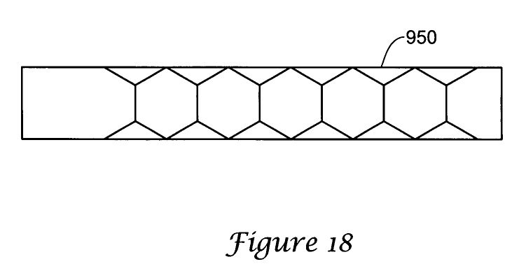

FIG. 18 illustrates a foldable or collapsible spinner tube 950. Spinner tube 950 can be made of a metal or polymer mesh. In other embodiments, spinner tube 950 is woven, knitted, braided, or made of intertwined metal or polymer fibers. The spinner tube 950 can be used as a delivery sleeve or retrieval sleeve.

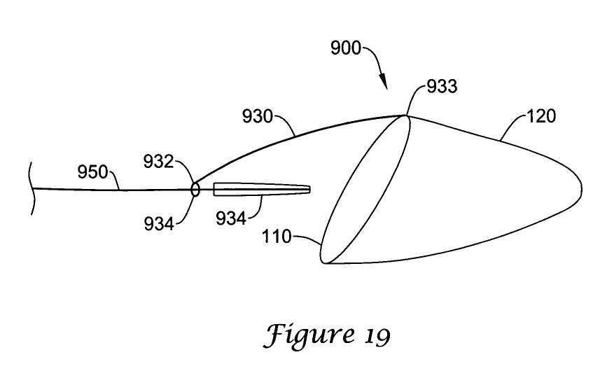

FIG. 19 shows a filter device 900 that is positioned distal of the elongate member 950. First end 933 of arm 930 is attached to frame 110 and second end 932 of arm has a slidable member 934 that slides over elongate member 950 and moves distally until it reaches stop 934 at distal end of elongate member 950. The length of arm 930 and position of stop 934 determines the position of filter device 900 within the vessel.

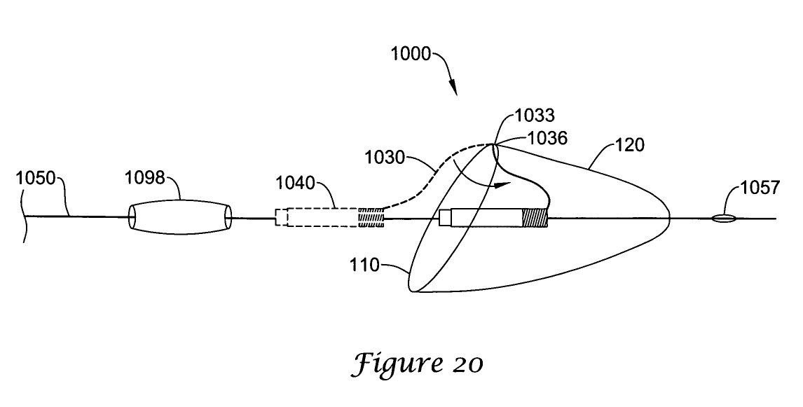

The filter device 1000 in FIG. 20 has a sliding attachment member 1040 connecting filter member 120 and arm 1030 to elongate member 1050. First end 1033 of arm 1030 is attached to frame 110 by a pressure sensitive hinge 1036. Sliding attachment member 1040 is moved by sliding pusher member 1098 distally along elongate member 1050. When pusher member 1098 contacts and pushes sliding attachment member 1040, it moves distally into the filter member 120. The pressure sensitive hinge 1036 may be incrementally moveable such that arm 1030 can be at any position from proximally extended through distally extended. In another embodiment, hinge 1036 snaps between a first position in which the arm 1030 is proximally extended and sliding attachment member 1040 is proximal of the filter member 120, and a second position in which the arm 1030 is distally extended into the filter member 120.

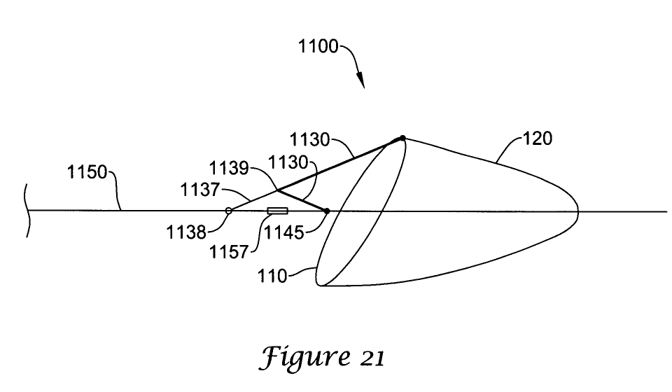

FIG. 21 shows a filter device 1100 having a bent, curved, or angled arm 1130 with tether 1137 connecting arm 1130 at the bend, curve or angle 1139 to elongate member 1150. Hard stop 1157 is disposed on elongate member 1150 between fixed attachment point 1145 of arm 1130 and moveable attachment point 1138 of tether 1137. In use, flexible tether 1137 allows a balloon catheter to slide over the tether 1137 and move close to filter device 1100. During retrieval, a retrieval sheath or sleeve is advanced over tether 1137 and bent arm 1130, collapsing filter 120 and frame 110.

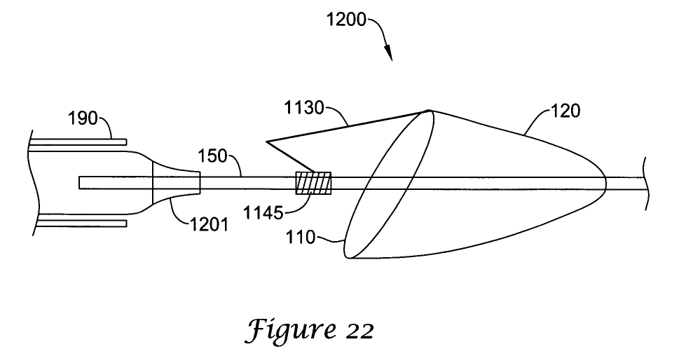

A modified bent arm filter device 1200 without a tether is illustrated in FIG. 22. Filter device 1200 includes filter member 120, frame 110, and elongate member 150. The bent arm 1130 is attached to elongate member 150 via attachment member 1145. In this device 1200, the bent arm 1130 allows a balloon tip 1201 to slide underneath arm 1130 and advance close to filter device 1200. During retrieval, a sheath or sleeve 190 is advanced over the bent arm 1130 to collapse the filter device 1200.

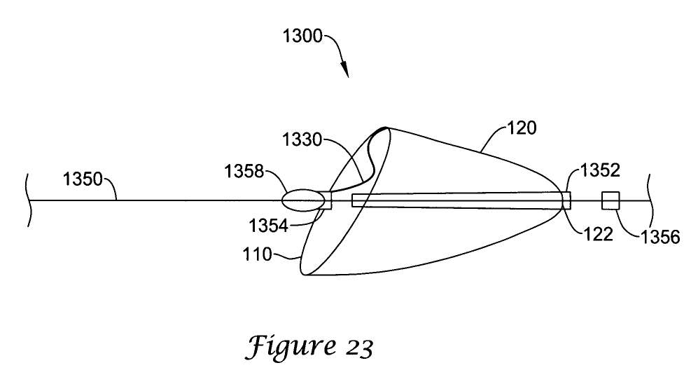

The filter device 1300 illustrated in FIG. 23 is disposed on split spinner tube 1352, 1354. The split spinner tube 1352, 1354 functions as the attachment member. Distal section 1352 of spinner tube is fixed to distal portion 122 of filter member and proximal section 1354 of spinner tube is fixed to arm 1330. Split spinner tube 1352, 1354 is disposed on guidewire 1350 between distal stop 1356 and fixed proximal stop 1358. Arm 1330 is fixed to proximal stop 1358. To retrieve filter device 1300, a retrieval sheath or sleeve is advanced distally over guidewire 1350 to arm 1330. As sleeve advances over arm 1330, filter member 120 and distal section 1352 of spinner tube move distally, collapsing filter member 120 and frame 110 against spinner tube 1352 and guidewire 1350.

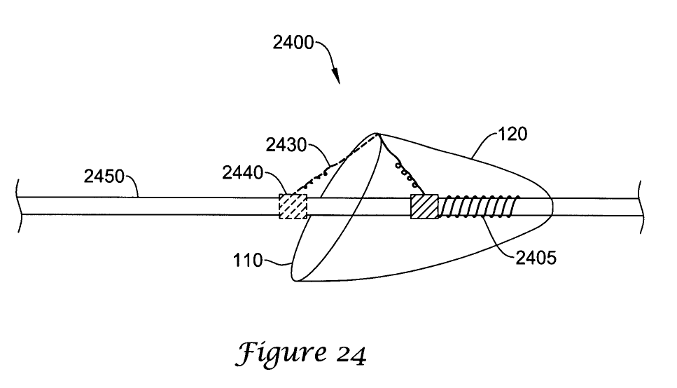

FIG. 24 shows filter device 2400 having spring arm 2430 fixed to frame 110 and sliding attachment member 2440 that slides on elongate member 2450. Spring 2405 is fixed to elongate member 2450 near the distal end thereof. Spring arm 2430 can be deflected by a balloon catheter, pushing sliding attachment member 2440 into filter member 120. As sliding attachment member 2440 moves distally, it contacts and contracts spring 2405, disposed around elongate member 2450. When the balloon catheter is withdrawn, spring 2405 expands, pushing sliding attachment member 2440 proximally to its equilibrium, or rest position.

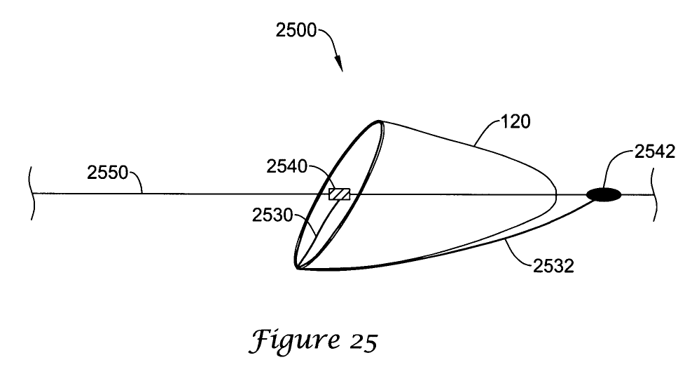

A dual arm filter device 2500 is shown in FIG. 25. First arm 2530 is fixed to frame 110 and sliding attachment member 2540. Second arm 2532 is fixed to frame 110 and attachment member 2542 located on elongate member 2550 distal of filter member 120.

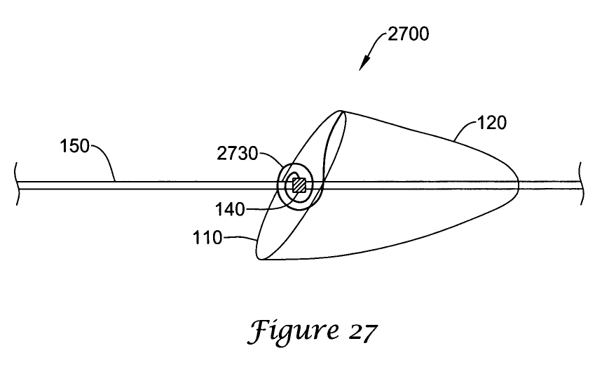

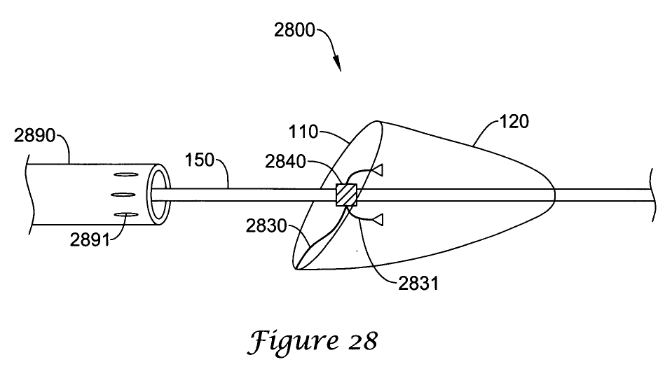

FIG. 27 illustrates a filter device 2700 with a spiral arm 2730 that circles elongate member 150. FIG. 28 illustrates a filter device 2800 with an attachment member 2840 having one or more fixing elements 2831, such as barbs, that mechanically engage retrieval sheath 2890. Retrieval sheath 2890 may have slots 2891 or other structure that mate with the fixing elements 2831. During retrieval, the retrieval sheath 2890 is advanced distally over elongate member 150 until the slots 2891 engage the fixing elements 2831, essentially locking the filter device 2800 onto the retrieval sheath 2890. Retrieval sheath 2890 is then withdrawn proximally, collapsing filter device 2800.

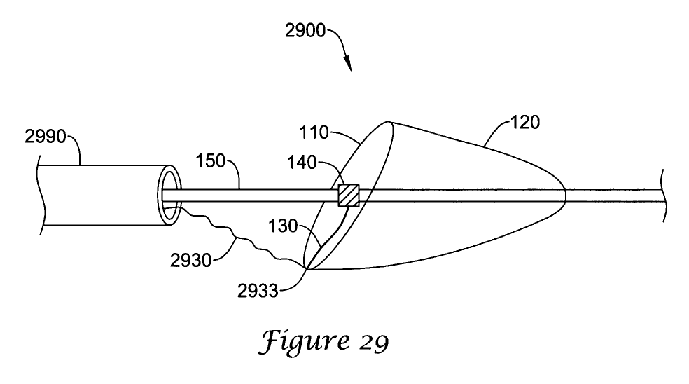

A filter device 2900 having a retrieval tether 2930 is illustrated in FIG. 29. Filter device 2900 includes filter member 120, frame 110, arm 130, attachment member 140 and elongate member 150, and retrieval tether 2930. The first end 2933 of tether 2930 is attached to frame 110 and the second end of tether 2930 extends along the elongate member 150. The tether 2930 is of sufficient length so as to be pulled through a hypotube 2990 or retrieval sheath during retrieval. The tether 2930 remains slack during deployment. During retrieval, a hypotube 2990 or retrieval sheath is advanced over elongate member 150 and tether 2930 to a position adjacent filter device 2900. The tether 2930 is pulled proximally, collapsing the frame 110 onto elongate member 150.

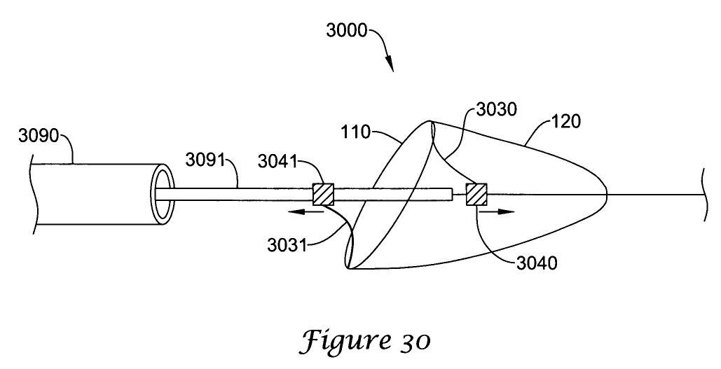

The filter device 3000 illustrated in FIG. 30 has a dual support arm assembly. The arms 3030, 3031 are attached at one end to frame 110 and attached at the other end to slidable attachment members 3040, 3041, which are disposed around and slide along elongate member 150. Proximal attachment member 3041 has a bore therethrough adapted to receive pusher 3091. During retrieval, a retrieval sleeve 3090 containing pusher 3091 is advanced over elongate member 150 towards filter device 3000. Pusher 3091 passes through the bore in proximal attachment member 3041 and pushes distal attachment member 3040 distally into filter member 120, thereby collapsing the filter device 3000 onto elongate member 150. The retrieval sleeve 3090 is advanced over the collapsed filter device 3000 and the sleeve 3090 containing collapsed filter device 3000 is withdrawn proximally from the vessel.

It should be noted that all of the devices according to the present invention can optionally be coated with an antithrombotic material, such as heparin (commercially available under the trade name Duraflow from Baxter) to inhibit clotting. Although the present invention has been described with reference to particular embodiments, workers skilled in the art will recognize that changes may be made in form and detail without departing from the spirit and scope of the invention.

1. An embolic protection device, comprising:
an elongate member having a proximal end and a distal end;
an expandable filter disposed about the elongate member, the filter having a proximal mouth portion facing the proximal end of the elongate member, and a distal portion extending toward to distal end of the elongate member; and
a support arm having a first end and second end; the first end coupled to the elongate member and the second end coupled to the mouth portion of the expandable filter; wherein when the filter is in an expanded orientation, the first end of the support arm is disposed at or distal of the mouth of the filter.

2. The embolic protection device of claim 1, wherein the filter is self-expanding.

3. The embolic protection device of claim 1, wherein the first end of the support arm is slidingly disposed on the elongate member.

4. The embolic protection device of claim 1, wherein the elongate member is a guidewire.

5. The embolic protection device of claim 1, wherein the expandable filter is supported at least at the mouth portion by a frame, wherein the frame defines the mouth of the filter.

6. The embolic protection device of claim 5, wherein the second end of the support arm is attached to the frame.

7. The embolic protection device of claim 1, wherein the first end of the support arm is disposed distal of the mouth of the filter.

8. The embolic protection device of claim 1, wherein the support arm is expandable and retractable.

9. The embolic protection device of claim 1, wherein the support arm is substantially perpendicular to the elongate member.

10. The embolic protection device of claim 1, wherein the support arm is made from a highly flexible, high tension strength material.

11. The embolic protection device of claim 1, wherein the support arm is shaped in appropriate curvature to facilitate apposition of the frame to the vessel wall and recovery by the retrieval member.

12. An intravascular filter device comprising:
an elongate member having a proximal region and a distal region;
at least one support arm attached to the elongate member at an attachment point;
a frame disposed about the elongate member and attached to the support arm such that the frame is spaced from the elongate member; and
a filter member having a proximal opening and a distal end, the proximal opening attached to the frame;
wherein the attachment point is in substantially the same axial space as the proximal opening of the filter member.

13. The filter device of claim 12, wherein the attachment point is distal of the proximal opening of the filter member.

14. The filter device of claim 12, wherein the support arm is attached to the elongate member via an attachment member.

15. The filter device of claim 14, wherein the attachment member is slidingly disposed on the elongate member.

16. The filter device of claim 14, wherein the support arm is moveable between a first position in which the attachment member is proximal of the frame, and a second position in which the attachment member is distal of the frame.

17. The filter device of claim 14, wherein the attachment member expands and contracts around the elongate member thereby reversibly holding and releasing the attachment member to the elongate member.

18. An emboli capturing system comprising:
an elongate member having a proximal end and a distal end;
an expandable filter disposed about the elongate member, the filter having:
a proximal mouth portion facing the proximal end of the elongate member, and a distal portion extending toward to distal end of the elongate member; and
at least one support arm having a first end and second end; the first end adapted to be coupled to the elongate member and the second end attached to the mouth portion of the expandable filter;
wherein the expandable emboli capturing device is moveable between a radially expanded position and a radially collapsed position, wherein when the filter is in an expanded orientation, the first end of the support arm is disposed at or distal of the mouth of the filter;
a retrieval member configured to be longitudinally moveable over the elongate member, the retrieval member having a receiving end configured to receive the filter in a collapsed position.

19. The emboli capturing system of claim 18, wherein the retrieval member includes an inner member adapted to engage the support arm thereby collapsing the filter.

20. The emboli capturing system of claim 19, wherein the inner member is a hollow tube.

21. The emboli capturing system of claim 18, wherein the support arm is coupled to the elongate member by an attachment member.

22. The filter device of claim 21, wherein the attachment member includes one or more fixing elements that mechanically engage the retrieval member.
